(19) United States
(12) Patent Application Publication
(10) Pub. No.: US 2021/0311050 A1
(43) Pub. Date: Oct. 7, 2021

(54) TARGETING PATHOGENIC B CELLS IN AUTOIMMUNITY

(71) Applicant: Research Foundation for the STATE UNIVERSITY OF NEW YORK, Albany, NY (US)

(72) Inventors: Gary Winslow, Syracuse, NY (US); Russell Levack, Tully, NY (US)

(21) Appl. No.: 17/265,099

(22) PCT Filed: Aug. 8, 2019
(86) PCT No.: PCT/US19/45624

§ 371 Date: Feb. 1, 2021

Related U.S. Application Data
(60) Provisional application No. 62/716,587, filed on Aug. 9, 2018.

(51) Int. Cl. G01N 33/569; A61K 31/7076

(52) U.S. Cl. CPC G01N 33/56972; G01N 2800/24; A61K 31/7076

(57) ABSTRACT

The present disclosure is directed to methods of treating a disease associated with pathogenic B cells comprising administering to a subject in need thereof an effective amount of an Adora2A (adenosine A2A receptor) agonist. The method more specifically comprises: (a) obtaining a biological sample from a subject, wherein the sample comprises B cells; (b) detecting in the sample the presence of pathogenic B cells; (c) administering to the subject a pharmaceutically effective dose of an Adora2A receptor agonist (A2aR agonist) if pathogenic B cells are detected in step (b). The method may further comprise administering to the subject subsequent doses of a pharmaceutically effective dose of the A2aR agonist until pathogenic B cells are no longer detected.

### CROSS REFERENCE TO RELATED APPLICATION

This application claims the benefit of priority from U.S. Provisional Application No. 62/716,587, filed Aug. 9, 2018, the entire contents of which are incorporated herein by reference.

### STATEMENT REGARDING FEDERALLY SPONSORED RESEARCH OR DEVELOPMENT

This invention was made with government support under Grant No. AI114545, awarded by the National Institutes of Health. The government has certain rights in the invention.

### BACKGROUND

Preventing and overcoming a pathogenic infection involves the coordinated cooperation of many differentiated immune cells. Memory B cells are one of many subtypes of B cells that participate in an adaptive immune response to effectively overcome an infection. The primary function of a memory B cell is to recognize a re-infection in the host and mount a more precise and accelerated immune response to the pathogen. This concept of immunological memory is essential in secondary immune responses, vaccinations, and chronic diseases.

A study in 2008 identified a novel B cell population in a bacterial infection model (Racine, R. et al., 2008, J Immunol, 181:1375-1385). This B cell population was identified by the unexpected expression of a surface marker known as CD11c. CD11c had been well-known as surface marker for dendritic cells, but had not been shown to be expressed by B cells. Two subsequent studies, by different groups, in 2012, identified a B cell population with identical characteristics in autoimmune patients, and in aged individuals (Rubtsov, A. V. et al., 2011, Blood, 118:1305-1311; Hao, Y. et al., 2011, Blood, 118:1294-1304). One of the studies also showed that the population expressed a transcription factor, T-bet; prior to that time, T-bet expression was characteristically associated with CD4 helper T cells. This novel B cell population is also referred to as Age-related B cells (ABCs), or T-bet⁺ B cells. Both T-bet and CD11c are useful markers for the identification of these B cells.

Studies in animals and in humans have shown that identical or equivalent cells are elicited during acute viral and parasitic infections (Barnett, B. E. et al., 2016, J Immunol, 197:1017-1022; Rubtsova, K. et al., 2013, Proc Nal Acad Sci USA, 110:E3216-3224; Krishnamurty, A. T. et al., 2016, Immunity, 45:402-414), but are also associated with autoimmune diseases, including Lupus (Karnell, J. L. et al., 2017, Cell Immunol, 321:40-45; Wang, S. et al., 2018, Nat Commun, 9:1758), and Crohn's disease (Wang, Z. et al., 2016, DNA Cell Biol, 35:628-635). Recent studies have shown that the frequency of T-bet+ B cells was increased in the peripheral blood of Systemic Lupus erythematosus (SLE) patients, relative to healthy controls (Wang, S. et al., 2018, Nat Commun, 9:1758). Some data suggest that this B cell subset includes cells that are pathogenic in autoimmune responses. Data from Liu et al. suggest that T-bet⁺ CD11c⁺ B cells contribute to the pathogenesis of lupus and provide a potential target for therapeutic intervention (Ya Liu et al., 2017 bioRxiv 116145; doi:10.1101/116145).

One study by Rubtsova et al. demonstrated that the T-bet⁺ B cell population was elicited in mice genetically-prone to SLE, and, importantly, elimination of the population by gene targeting ameliorated disease (Rubtsova, K. et al., 2017, J Clin Invest, 127:1392-1404). This seminal study demonstrated that T-bet⁺ B cell population plays an important role in autoimmunity, and suggested that drug targeting designed to reduce the number of T-bet+ B cells may provide an effective treatment for autoimmune disease.

The inventors have previously demonstrated that T-bet⁺ B cells can function as memory B cells (Kenderes, K. J. et al., 2018, Cell Rep, 24:824-837 e823; Yates, J. L. et al., 2013, J Immunol, 191:1240-1249). On the basis of their findings, and their observation that the cells identified during bacterial infection were identical to those identified in autoimmunity, the inventors concluded that the T-bet⁺ B cells can function in both immunity and immunopathology (Winslow, G. M. et al., 2017, Cell Immunol, 321:8-17). This implies that studies of T-bet⁺ B cells elicited by infection have direct relevance to T-bet⁺ B cells in the context of other human diseases.

Systemic lupus erythematosus (SLE) is a relapsing autoimmune disease that affects approximately 1.5 million people in the US, and 5 million worldwide. The disease is highly variable, but commonly is manifested by facial rash, fatigue, joint pain, and disease flares, followed in some cases by end-stage renal disease and organ failure. Autoantibodies, in particular anti-nuclear antibodies (ANAs), are characteristic of the disease, implicating B cells and T cells as major mediators. Although there are many drugs for SLE in use, or under development, none are completely effective, and many lack sufficient specificity. Thus, there is an urgent need for new treatments to limit and/or reverse disease.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1C. An adenosine receptor agonist eliminated CD11c⁺ T-bet⁺ B cells from the spleens of infected mice. C57BL/6 mice were infected with E. muris on day 0; thirty days later the mice were administered DMSO (vehicle control), or the adenosine A2 receptor agonist CGS21680 (2.5 mg/kg, daily, for 7 days). Spleen cells were harvested on day 37 and analyzed for the presence of CD11c⁺ T-bet⁺ B cells (green arrow in control plot). In mice that received the agonist treatment, the CD11c⁺ B cells were largely absent. Representative flow cytometry dot plots are shown in (A) Vehicle Control, and (B) Adenosine Agonist (CGS21680). (C) Aggregate data of the experiments shown in (A) and (B). ***=p<0.0001.

FIGS. 2A-2C. Agonist treatment resulted in prolonged depletion of T-bet⁺ CD11c+ B cells. Groups of five C57BL/6 mice were infected with E. muris on day 0; thirty days later the mice were administered DMSO (vehicle control), or adenosine A2 receptor agonist CGS21680 (2.5 mg/kg, daily, for 7 days). Spleen cells were harvested 12 days following cessation of treatment (on day 48) and were analyzed for the presence of CD11c⁺ T-bet⁺ B cells (green arrow in control plot). Representative flow cytometry dot plots are shown in (A) Vehicle Control, and (B) Adenosine Agonist (CGS21680). (C) Aggregate data of the experiments shown in (A) and (B). ***=p<0.0001.

FIGS. 3A-3B. Agonist treatment did not eliminate T-bet⁺ B cells that lacked expression of the A2aR. CD11c and CD19 flow cytometry data from (A) Wild type mice (B) mice that lacked A2aR only on B cells. The mice were infected with E. muris; 30 days post-infection wild-type or MB1-cre×A2aR^{fl/fl} (B cell-specific A2aR KO) mice were treated with CGS21680 every other day for 7 days, at which time spleen cells were analyzed by flow cytometry. CD11c⁺ CD19⁺ B cells (also T-bet⁺) were eliminated in the wild-type mice (A), but not in the B cell-specific A2aR KO mice (B). The data shown are representative of several experiments. These data demonstrate that the agonist acts directly on B cells.

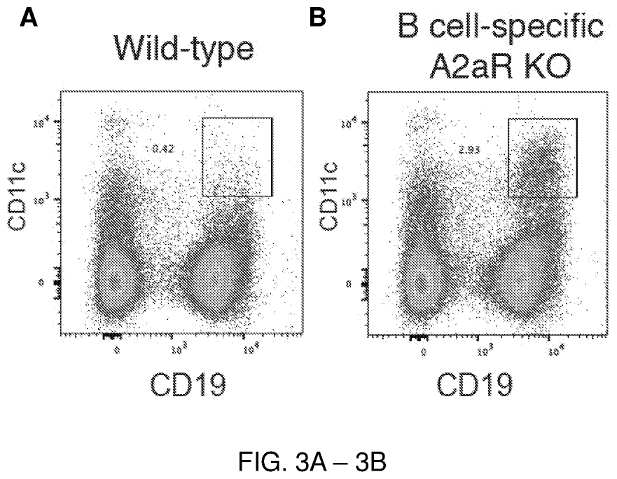

FIGS. 4A-4D. Agonist treatment eliminated T-bet+ B cells in lupus-prone SLE1.2.3 (B6.NZMs^{Sle1/Sle2/Sle3}) mice. (A) CD11c and CD19 flow cytometry data from vehicle treated mice. (B) CD11c and CD19 flow cytometry data from A2aR agonist (CGS21680)-treated mice. (C) T-bet and CD19 flow cytometry data from vehicle-treated mice. (D) T-bet and CD19 flow cytometry data from agonist (CGS21680)-treated mice. Nine-month old SLE mice were treated with vehicle, or CGS21680, every other day for 7 days, at which time spleen cells were analyzed by flow cytometry. CD11c⁺ CD19⁺ ((A) and (B)) or T-bet⁺ CD19⁺ ((C) and (D)) B cells were analyzed. B cells were largely eliminated in the agonist-treated SLE1.2.3 mice. The data shown are representative of an experiment where n=6.

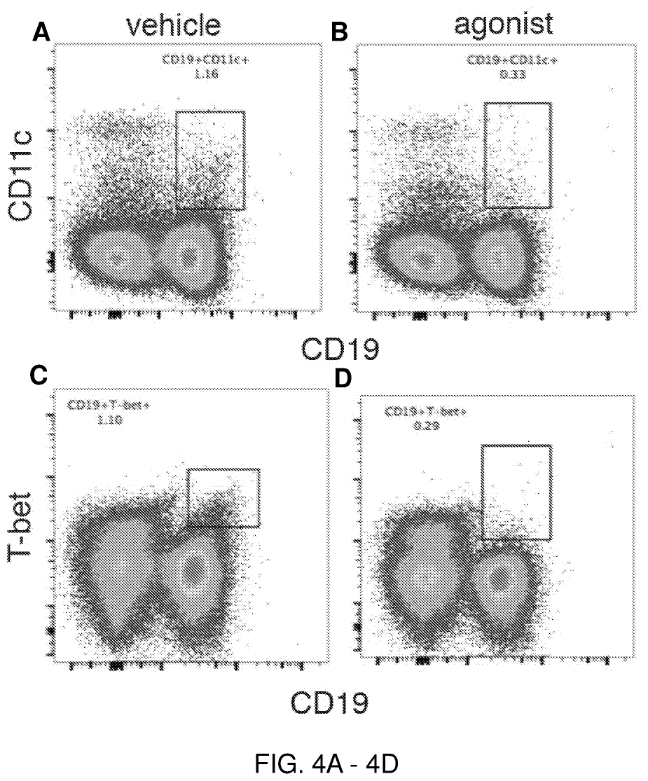

FIGS. 5A-5B. Agonist treatment eliminated T-bet⁺ B cells in lupus-prone MRL/lpr (MRL/MpJ-Fas^{lpr}/J) mice. CD11c and CD19 flow cytometry data from (A) vehicle-treated mice and (B) A2aR agonist (CGS21680)-treated mice. 12 to 13 week old MRL/lpr mice were treated with vehicle, or CGS21680, every other day for 7 days, at which time spleen cells were analyzed by flow cytometry. Most of the CD11c⁺ CD19⁺ B cells were eliminated in the agonist-treated mice.

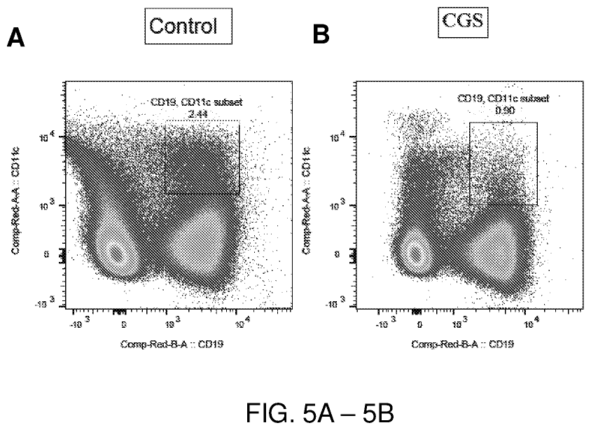

FIGS. 6A-6B. In vitro agonist treatment eliminated T-bet⁺ B cells. CD11c and CD19 flow cytometry data using B cells isolated from (A) vehicle-treated splenocytoes and (B) A2aR agonist (CGS21680)-treated splenocytes. C57BL/6 mice were infected with E. muris; 30 days post-infection, T cell-depleted spleen cells were incubated in cell culture medium treated with vehicle, or CGS21680 (1 μM), for 48 hours. Approximately 50% of the CD11c⁺ CD19⁺ B cells were depleted by the agonist.

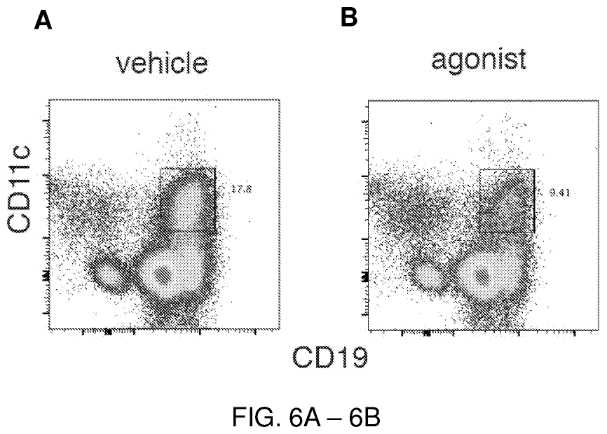

FIGS. 7A-7B. In vitro Regadenoson treatment eliminated T-bet⁺ B cells. (A) vehicle treated splenocytes and (B) agonist (Regadenoson)-treated splenocytes. Mice were infected with E. muris; 44 days post-infection, T cell-depleted spleen cells were incubated in medium with or without Regadenson (10 μM), for 48 hours. Approximately 30% of the CD11c⁺ CD19⁺ B cells were depleted by Regadenoson in vitro.

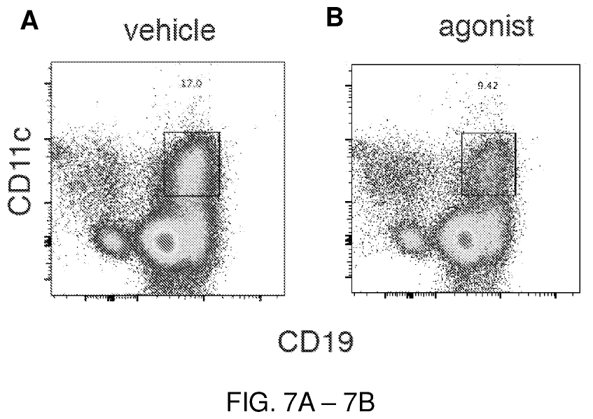

FIG. 8A-8C. T-bet+ Age-related B cells (ABCs) were depleted in aged mice upon CGS21680 treatment. CD11c and CD19 flow cytometry data from (A) vehicle-treated and (B) A2aR agonist-(CGS21680) treated aged mice. CGS21680 was administered at day 0, 4 and 6. Mice were sacrificed on day 7 and ABCs were identified using flow cytometry. (C) Quantification of the data in (A) and (B).

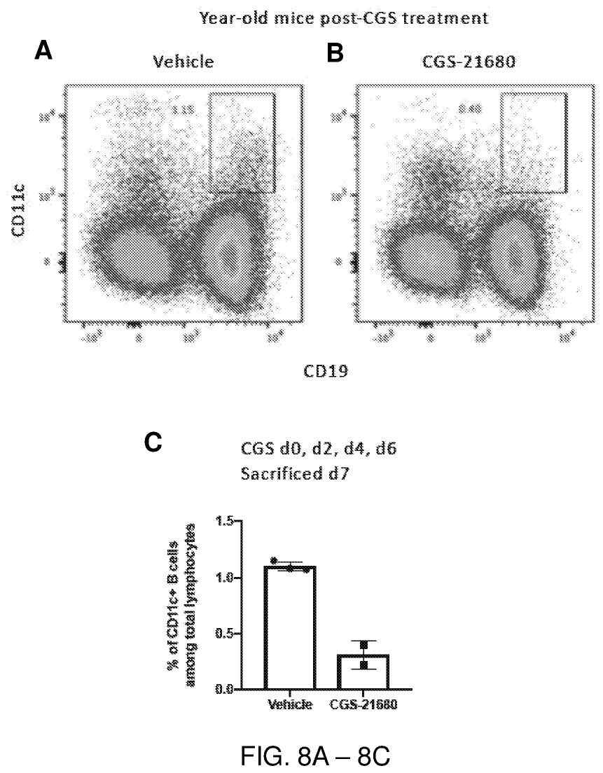

FIG. 9. CGS21680 was administered to lupus-prone MRL/lpr mice, starting at 9 weeks of age (lupus-prone MRL/lpr mice typically exhibit disease symptoms at about 15 weeks), once weekly, and the mice were sacrificed at 15 weeks of age. CGS21680-treated mice had much lower amounts of anti-nuclear antibodies (ANAs). ANAs are a signature of disease in lupus mice.

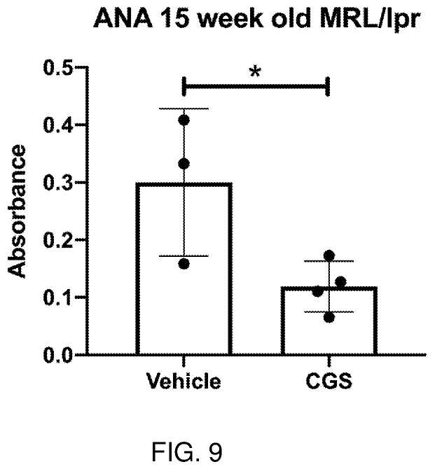

FIGS. 10A-10C. Regadenoson treatment reduced the number of Tbet⁺ B cells in E. muris-infected mice. Starting day 30 post-infection, C57BL6/J mice were treated with (A) vehicle (B) or Regadenoson (2.5 mg/kg) every other day for 7 days. Splenocytes were analyzed by flow cytometry on day 37 post-infection. (C) A unpaired, two-tailed Student's t-test was used to determine significance (P=0.0049).

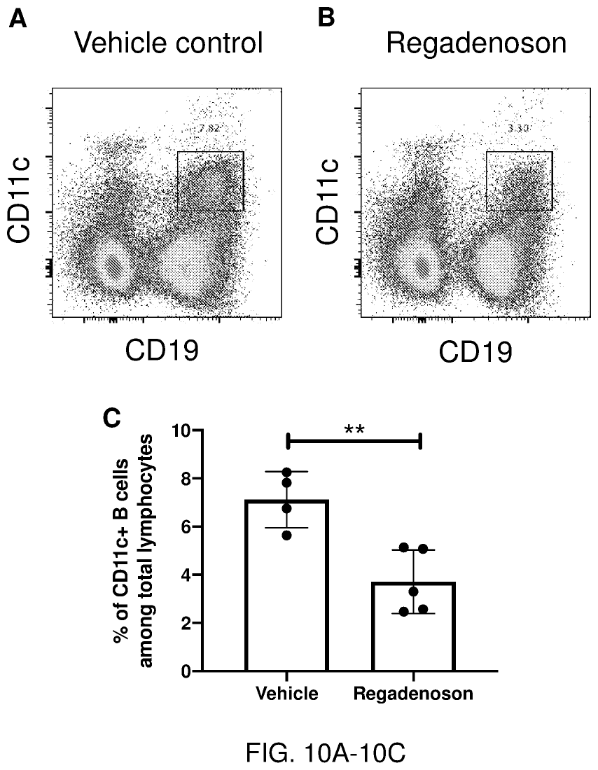

### DETAILED DESCRIPTION

### Definitions

As used herein, the term “about” refers to an approximately ±10% variation from a given value.

The term “detecting” is used in the broadest sense to include both qualitative and quantitative measurements of a target molecule in a biological sample (e.g., a serum sample).

The term “biological sample” includes body samples from an animal, including biological fluids such as serum, plasma, vitreous fluid, lymph fluid, synovial fluid, follicular fluid, seminal fluid, amniotic fluid, milk, whole blood, urine, cerebro-spinal fluid, saliva, sputum, tears, perspiration, mucus, and tissue culture medium, as well as tissue extracts such as homogenized tissue, and cellular extracts. In some embodiments, the biological sample is a serum, plasma or urine sample.

In some embodiments, the term “hydrocarbon group” (also denoted by the group R) is defined as a chemical group composed solely of carbon and hydrogen, except that the hydrocarbon group may (i.e., optionally) be substituted with one or more fluorine atoms to result in partial or complete fluorination of the hydrocarbon group. In different embodiments, one or more of the hydrocarbon groups can contain, for example, 1, 2, 3, 4, 5, 6, 7, 8, 9, 10, 11, 12, 13, 14, 15, 16, 17, 18, 19, or 20 carbon atoms, or a number of carbon atoms within a particular range bounded by any two of the foregoing carbon numbers (e.g., 1-6 carbon atoms). Hydrocarbon groups in different compounds described herein, or in different positions of a compound, may possess the same or different number (or preferred range thereof) of carbon atoms in order to independently adjust or optimize its efficacy or physical properties.

In a first set of embodiments, the hydrocarbon group (R) is a saturated and straight-chained group, i.e., a straight-chained (linear) alkyl group. Some examples of straight-chained alkyl groups include methyl, ethyl, n-propyl, n-butyl, n-pentyl, n-hexyl, n-heptyl, n-octyl, n-nonyl, n-decyl, n-undecyl, n-dodecyl, n-tridecyl, n-tetradecyl, n-pentadecyl, n-hexadecyl, n-heptadecyl, n-octadecyl, and n-eicosyl groups.

In a second set of embodiments, the hydrocarbon group (R) is saturated and branched, i.e., a branched alkyl group. Some examples of branched alkyl groups include isopropyl (2-propyl), isobutyl (2-methylprop-1-yl), sec-butyl (2-butyl), t-butyl (1,1-dimethylethyl-1-yl), 2-pentyl, 3-pentyl, 2-methylbut-1-yl, isopentyl (3-methylbut-1-yl), 1,2-dimethylprop-1-yl, 1,1-dimethylprop-1-yl, neopentyl (2,2-dimethylprop-1-yl), 2-hexyl, 3-hexyl, 2-methylpent-1-yl, 3-methylpent-1-yl, isohexyl (4-methylpent-1-yl), 1,1-dimethylbut-1-yl, 1,2-dimethylbut-1-yl, 2,2-dimethylbut-1-yl, 2,3-dimethylbut-1-yl, 3,3-dimethylbut-1-yl, 1,1,2-trimethylprop-1-yl, 1,2,2-trimethylprop-1-yl groups, isoheptyl, isooctyl, and the numerous other branched alkyl groups having up to 20 carbon atoms, wherein the “1-yl” suffix represents the point of attachment of the group.

In a third set of embodiments, the hydrocarbon group (R) is saturated and cyclic, i.e., a cycloalkyl group. Some examples of cycloalkyl groups include cyclopropyl, cyclobutyl, cyclopentyl, cyclohexyl, cycloheptyl, and cyclooctyl groups. The cycloalkyl group can also be a polycyclic (e.g., bicyclic) group by either possessing a bond between two ring groups (e.g., dicyclohexyl) or a shared (i.e., fused) side (e.g., decalin and norbornane).

In a fourth set of embodiments, the hydrocarbon group (R) is unsaturated and straight-chained, i.e., a straight-chained (linear) olefinic or alkenyl group. The unsaturation occurs by the presence of one or more carbon-carbon double bonds and/or one or more carbon-carbon triple bonds. Some examples of straight-chained olefinic groups include vinyl, propen-1-yl (allyl), 3-buten-1-yl (CH₂═CH—CH₂—CH₂—), 2-buten-1-yl (CH₂—CH═CH—CH₂—), butadienyl, 4-penten-1-yl, 3-penten-1-yl, 2-penten-1-yl, 2,4-pentadien-1-yl, 5-hexen-1-yl, 4-hexen-1-yl, 3-hexen-1-yl, 3,5-hexadien-1-yl, 1,3,5-hexatrien-1-yl, 6-hepten-1-yl, ethynyl, propargyl (2-propynyl), 3-butynyl, and the numerous other straight-chained alkenyl or alkynyl groups having up to 20 carbon atoms.

In a fifth set of embodiments, the hydrocarbon group (R) is unsaturated and branched, i.e., a branched olefinic or alkenyl group. Some examples of branched olefinic groups include propen-2-yl (CH₂═C.—CH₃), 1-buten-2-yl (CH₂═C.—CH₂—CH₃), 1-buten-3-yl (CH₂═CH—CH.—CH₃), 1-propen-2-methyl-3-yl (CH₂═C(CH₃)—CH₂—), 1-penten-4-yl, 1-penten-3-yl, 1-penten-2-yl, 2-penten-2-yl, 2-penten-3-yl, 2-penten-4-yl, and 1,4-pentadien-3-yl, and the numerous other branched alkenyl groups having up to 20 carbon atoms, wherein the dot in any of the foregoing groups indicates a point of attachment.

In a sixth set of embodiments, the hydrocarbon group (R) is unsaturated and cyclic, i.e., a cycloalkenyl group. The unsaturated cyclic group can be aromatic or aliphatic. Some examples of unsaturated cyclic hydrocarbon groups include cyclopropenyl, cyclobutenyl, cyclopentenyl, cyclopentadienyl, cyclohexenyl, cyclohexadienyl, phenyl, benzyl, cycloheptenyl, cycloheptadienyl, cyclooctenyl, cyclooctadienyl, and cyclooctatetraenyl groups. The unsaturated cyclic hydrocarbon group may or may not also be a polycyclic group (such as a bicyclic or tricyclic polyaromatic group) by either possessing a bond between two of the ring groups (e.g., biphenyl) or a shared (i.e., fused) side, as in naphthalene, anthracene, phenanthrene, phenalene, or indene fused ring systems.

In other embodiments, the hydrocarbon group (R) includes (i.e., is substituted with) at least one heteroatom (i.e., non-carbon and non-hydrogen atom), such as one or more heteroatoms selected from oxygen, nitrogen, sulfur, and halide atoms, as well as groups containing one or more of these heteroatoms (i.e., heteroatom-containing groups). In some embodiments, the hydrocarbon group does not contain hydrogen atoms (e.g., where all hydrogen atoms are replaced with heteroatoms, such as in —CF₃), while in other embodiments, the hydrocarbon group contains at least one hydrogen atom. Some examples of oxygen-containing groups include hydroxy (OH), alkoxy (OR), carbonyl-containing (e.g., carboxylic acid, ketone, aldehyde, carboxylic ester, amide, and urea functionalities), nitro (NO₂), carbon-oxygen-carbon (ether), sulfonyl, and sulfinyl (i.e., sulfoxide) groups. Some particular examples of alkoxy groups (—OR) include methoxy, ethoxy, n-propoxy, isopropoxy, n-butoxy, isobutoxy, t-butoxy, phenoxy, benzyloxy, 2-hydroxyethoxy, 2-methoxyethoxy, 2-ethoxyethoxy, vinyloxy, and allyloxy groups. In the case of an ether group, the ether group can also be a polyalkyleneoxide (polyalkyleneglycol) group, such as a polyethyleneoxide group. Some examples of nitrogen-containing groups include primary amine, secondary amine, tertiary amine (i.e., —NR′₂ or NR′₃+, wherein R′ is independently selected from H and hydrocarbon groups set forth above), nitrile (CN), amide (i.e., —C(O)NR′₂ or —NRC(O)R′, wherein R′ is independently selected from hydrogen atom and hydrocarbon groups set forth above), imine (e.g., —CR′═NR′, wherein R′ is independently H or a hydrocarbon group), urea (—NR′—C(O)—NR′₂, wherein R′ is independently H or a hydrocarbon group), and carbamate groups (—NR′—C(O)—OR′, wherein R′ is independently H or a hydrocarbon group). Some examples of sulfur-containing groups include mercapto (i.e., —SH), thioether (i.e., sulfide, e.g., —SR), disulfide (—R—S—S—R), sulfoxide (—S(O)R), sulfone (—SO₂R), sulfonate (—S(═O)₂OR″, wherein R″ is H, a hydrocarbon group, or a cationic group), and sulfate groups (—OS(═O)₂OR″, wherein R″ is H, a hydrocarbon group, or a cationic group). Some examples of halide atoms include fluorine, chlorine, bromine, and iodine. One or more of the heteroatoms described above (e.g., oxygen, nitrogen, and/or sulfur atoms) can be inserted between carbon atoms (e.g., as —O—, —NR′—, or —S—) in any of the hydrocarbon groups described above. Alternatively, or in addition, one or more of the heteroatom-containing groups can replace one or more hydrogen atoms on the hydrocarbon group. In some embodiments, any one or more of the above groups is excluded.

### General Description

In some embodiments, the instant disclosure is directed to methods of treating a disease manifested by the presence of pathogenic B cells by administering a subject in need thereof an effective amount of an Adora2A (adenosine A2A receptor) agonist (A2aR agonist).

In some embodiments, the method of treating comprises (a) obtaining a biological sample from a subject, wherein the sample comprises B cells; (b) detecting in the sample the presence of pathogenic B cells; (c) administering to the subject a pharmaceutically effective dose of an Adora2A (adenosine A2A receptor) agonist (A2aR agonist) if pathogenic B cells are detected in step (b).

In some embodiments, the method of treating further comprises (d) obtaining, after the administering in step (c), an additional biological sample from a subject, wherein the additional biological sample comprises B cells; (e) detecting in the additional biological sample the presence of pathogenic B cells; and (f) administering to the subject a pharmaceutically effective dose of the A2aR agonist if pathogenic B cells are detected in step (e).

In some embodiments, the method of treatment is continued until no or few pathogenic B cells can be detected in the subject's biological sample.

In some embodiments, pathogenic B cells comprise T-bet⁺ B cells (i.e., B cells that express the T-bet transcription factor). In some embodiments, the pathogenic B cells comprise CD11c⁺ cells (i.e., B cells that express CD11c (aka. Integrin, alpha X (complement component 3 receptor 4 subunit) (ITGAX)). In some embodiments, the pathogenic B cells comprise T-bet⁺ and CD11c⁺ double-positive B cells. In some embodiments, the pathogenic B cells comprise T-bet⁺ CD11c-negative cells. In some embodiments, the pathogenic B cells further comprise CD11b⁺ cells. In some embodiments, the pathogenic B cells further comprise CD73⁺ cells. In some embodiments, the pathogenic B cells further comprise PD-L2⁺ cells.

In some embodiments, the detection of pathogenic B cells is achieved by flow cytometry. In some embodiments, antibodies against T-bet are used in the detection. In some embodiments, antibodies against CD11 are used in the detection. In a specific embodiment, antibodies against CD11c are used in the detection. In some embodiments, antibodies against CD11b are used in the detection. In some embodiments, antibodies against CD73 are used in the detection. In some embodiments, antibodies against PD-L2 are used in the detection. In some embodiments, the detection antibodies are polyclonal antibodies. In some embodiments, the detection antibodies are monoclonal antibodies.

In some embodiments, pathogenic B cells are detected using antibodies against a single cellular marker. In a specific embodiment, the single marker is T-bet. In a specific embodiment, the single marker is CDC11c. In some embodiments, pathogenic B cells are detected using a combination of antibodies, wherein each group of antibody within the combination is directed to a different marker. In some embodiments, pathogenic B cells are detected using a combination comprising T-bet and CDC11c antibodies. In some embodiments, the combination further comprises an antibody selected from the group consisting of CD11b, CD73 and PD-L2.

In some embodiments, the diseases manifested by the presence of pathogenic B cells include, but are not limited to, autoimmune diseases. In some embodiments, the diseases manifested by the presence of pathogenic B cells comprise any autoimmune disease wherein these cells have or may be implicated in pathogenesis.

In a specific embodiment, the autoimmune disease is lupus (systemic lupus erythematosus; SLE). In a specific embodiment, the autoimmune disease is Chron's disease. In a specific embodiment, the autoimmune disease is arthritis. In a specific embodiment, the autoimmune disease is multiple sclerosis.

In a specific embodiment, the disease manifested by the presence of pathogenic B cells is common variable immunodeficiency (CVID) with autoimmune cytopenia.

In some embodiments, the disease manifested by the presence of pathogenic B cells is Sjogren's syndrome. In a specific embodiment, the disease manifested by the presence of pathogenic B cells is Sjogren's syndrome-associated lymphoproliferation.

In some embodiments, a A2aR agonist is combined with a pharmaceutically acceptable carrier prior to administration. For the purposes of this disclosure, “pharmaceutically acceptable carriers” means any of the standard pharmaceutical carriers. Examples of suitable carriers are well known in the art and may include, but are not limited to, any of the standard pharmaceutical carriers such as a phosphate buffered saline solution and various wetting agents. Other carriers may include additives used in tablets, granules and capsules, and the like. Typically such carriers contain excipients such as starch, milk, sugar, certain types of clay, gelatin, stearic acid or salts thereof, magnesium or calcium stearate, talc, vegetable fats or oils, gum, glycols or other known excipients. Such carriers may also include flavor and color additives or other ingredients. Compositions comprising such carriers are formulated by well-known conventional methods. In some embodiments, a carrier is not used or required.

An A2aR agonist can be admixed with a pharmaceutically acceptable carrier to make a pharmaceutical preparation in any conventional form including, inter alia, a solid form such as tablets, capsules (e.g. hard or soft gelatin capsules), pills, cachets, powders, granules, and the like; a liquid form such as solutions, suspensions; or in micronized powders, sprays, aerosols and the like.

In some embodiments, a composition of the present disclosure can be administered by different routes of administration, such as an oral, oronasal, or parenteral route. In particular embodiments, the agonist is administered intravenously.

“Oral” or “peroral” administration refers to the introduction of a substance into a subject's body through or by way of the mouth and involves swallowing or transport through the oral mucosa (e.g., sublingual or buccal absorption) or both.

“Oronasal” administration refers to the introduction of a substance into a subject's body through or by way of the nose and the mouth, as would occur, for example, by placing one or more droplets in the nose. Oronasal administration involves transport processes associated with oral and intranasal administration.

“Parenteral administration” refers to the introduction of a substance into a subject's body through or by way of a route that does not include the digestive tract. Parenteral administration includes subcutaneous administration, intramuscular administration, transcutaneous administration, intradermal administration, intraperitoneal administration, intraocular administration, and intravenous administration.

In some embodiments, compositions comprising an A2aR agonist can be administered by aerosol. For example, this can be accomplished by preparing an aqueous aerosol, liposomal preparation or solid particles containing a composition comprising an A2aR agonist preparation. A nonaqueous (e.g., fluorocarbon propellant) suspension could be used. Sonic nebulizers can also be used. An aqueous aerosol is made by formulating an aqueous solution or suspension of the agent together with conventional pharmaceutically acceptable carriers and stabilizers.

Typically, an effective amount of an A2aR agonist is about 0.01 mg/kg to 100 mg/kg (wherein “mg/kg” indicates “mg per kg body weight”. In different embodiments, the effective amount of an A2aR agonist is precisely, about, or at least, for example, 0.01 mg/kg, 0.05 mg/kg, 0.1 mg/kg, 0.2 mg/kg, 0.5 mg/kg, 1 mg/kg, 2 mg/kg, 5 mg/kg, 8 mg/kg, 10 mg/kg, 20 mg/kg, 30 mg/kg, 40 mg/kg, 50 mg/kg, 60 mg/kg, 70 mg/kg, 80 mg/kg, 90 mg/kg, 100 mg/kg, 150 mg/kg, 175 mg/kg or 200 mg/kg of A2aR agonist, or an amount within a range bounded by any two of the foregoing values. In some embodiments, an effective amount of an A2aR agonist is less than 0.2 mg/kg, e.g., about 0.15 mg/kg, 0.1 mg/kg, 0.05 mg/kg, or 0.01 mg/kg. In some embodiments, the A2aR agonist is administered to a patient in need thereof every day, every other day, every three days, or once a week, for a suitable time period, e.g., one, two, three, four, five, six, seven, or eight weeks, or indefinitely until no pathogenic B cells are detected from biological samples of the patient.

### A2aR Agonists

In some embodiments, the A2aR agonist is a small molecule compound. The term “small molecule compound” herein refers to a small organic chemical compound, generally having a molecular weight of up to or less than 5000 daltons, 2000 daltons, 1500 daltons, 1000 daltons, 800 daltons, or 600 daltons.

In some embodiments, the A2aR agonist has the following chemical structure:

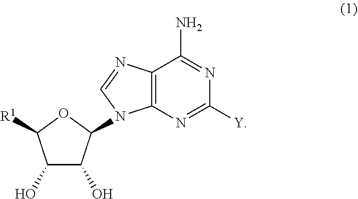

In Formula (1) above, R¹ may be —CH₂OH, —CO₂H, —CO₂R², —C(O)NR³R⁴, or —C(O)NHR⁴, wherein R², R³, and R⁴ are independently hydrocarbon groups (R) containing 1, 2, 3, 4, 5, or 6 carbon atoms (e.g., 1-3, 1-4, or 1-6 carbon atoms), as described above. In particular embodiments, R², R³, and R⁴ are linear or branched alkyl groups containing 1-6, 1-5, 1-4, 1-3, or 1-2 carbon atoms. Typically, the hydrocarbon group in R², R³, and R⁴ is not substituted, i.e., contains only carbon and hydrogen atoms. In Formula (1), Y may be R⁵, —OR⁶, —NR⁷R⁸, —NHR⁸, —NH—N═CR⁹R¹⁰, or —NH—N═CHR¹⁰, wherein R⁵, R⁶, R⁷, R⁸, R⁹, and R¹⁰ are independently hydrocarbon groups containing 1-20 carbon atoms (or sub-grouping of carbon numbers therein), as described above, and optionally substituted with one or more heteroatoms, such as nitrogen and oxygen atoms.

In particular embodiments, Y is —NR⁷R⁸ or —NHR⁸, or more particularly, Y is —NHR⁸. In further embodiments, R⁸ contains a ring, such as a five-membered or six-membered ring, as described above. The ring may be, for example, a benzene ring. In specific embodiments, R⁸ has the following structure:

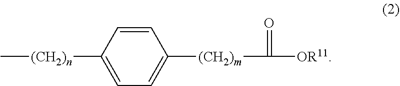

wherein n and m are independently integers of 1, 2, and 3, and R¹¹ is a hydrogen atom or a hydrocarbon group (or more specifically, alkyl group) containing 1-6, 1-5, 1-4, 1-3, or 1-2 carbon atoms.

In some embodiments, the A2 receptor agonist is CGS21680, or a derivative thereof with the following chemical formula:

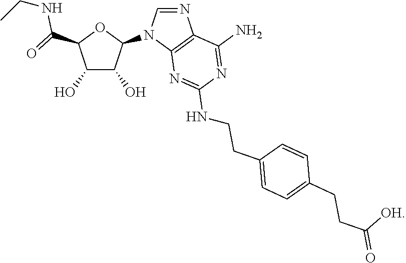 [SMILES: CCNC(=O)[C@H]1O[C@@H](n2cnc3c(N)nc(NCCc4ccc(CCC(=O)O)cc4)nc32)[C@H](O)[C@@H]1O]

In some embodiments, the A2 receptor agonist is ATL-146e (Apadenoson, STREDAVASE™) (described in Jacobson K A et al., 2006, Nature Reviews. Drug Discovery, 5 (3): 247-64), which has the following chemical structure:

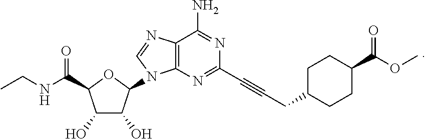

In some embodiments, the A2 receptor agonist is YT-146 (2-(1-octynyl)adenosine) (Yoneyama F. et al., 1992, European Journal of Pharmacology, 213 (2): 199-204), which has the following chemical structure:

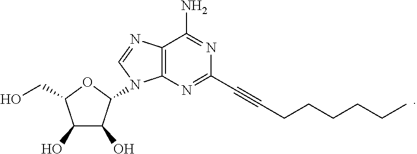 [SMILES: CCCCCCC#Cc1nc(N)c2ncn([C@H]3O[C@@H](CO)[C@H](O)[C@@H]3O)c2n1]

In some embodiments, the A2 receptor agonist is DPMA (N6-(2-(3,5-dimethoxyphenyl)-2-(2-methylphenyl)ethyl)adenosine) (described in Jacobson K A et al., 2006, Nature Reviews. Drug Discovery, 5 (3): 247-64), which has the following chemical structure:

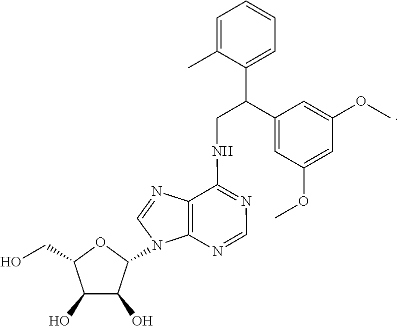 [SMILES: COc1cc(OC)cc(C(CNc2ncnc3c2ncn3[C@H]2O[C@@H](CO)[C@H](O)[C@@H]2O)c2ccccc2C)c1]

In some embodiments, the A2 receptor agonist is Regadenoson (i.e., Lexiscan, CV-3146), which has the following chemical structure:

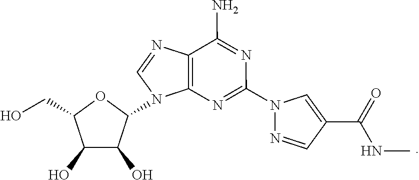 [SMILES: CNC(=O)c1cnn(-c2nc(N)c3ncn([C@H]4O[C@@H](CO)[C@H](O)[C@@H]4O)c3n2)c1]

In some embodiments, the A2 receptor agonist is UK-432,097, which has the following chemical structure:

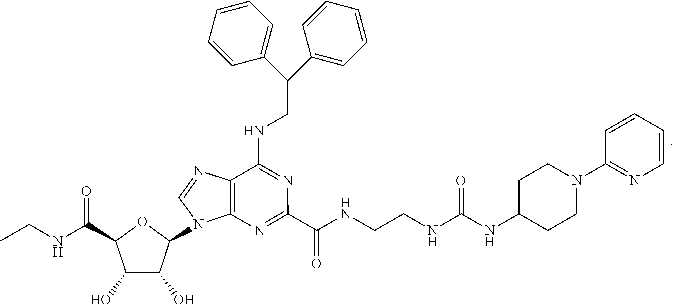 [SMILES: CCNC(=O)[C@H]1O[C@@H](n2cnc3c(NCC(c4ccccc4)c4ccccc4)nc(C(=O)NCCNC(=O)NC4CCN(c5ccccn5)CC4)nc32)[C@H](O)[C@@H]1O]

In some embodiments, the A2 receptor agonist is limonene, which has the following chemical structure:

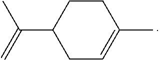

In some embodiments, the A2 receptor agonist is Zeatin riboside, which has the following chemical structure:

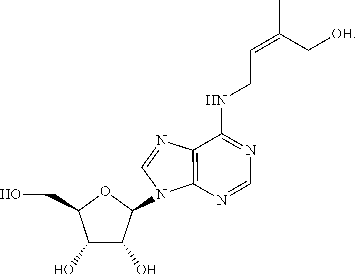 [SMILES: C/C(=C/CNc1ncnc2c1ncn2[C@@H]1O[C@H](CO)[C@@H](O)[C@H]1O)CO]

In some embodiments, the A2 receptor agonist is NECA (5′-(N-ethylcarboxamido)adenosine) (described in Jacobson K A et al., 2006, Nature Reviews. Drug Discovery, 5 (3): 247-64), which has the following chemical structure:

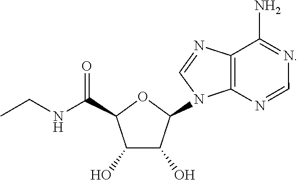 [SMILES: CCNC(=O)[C@H]1O[C@@H](n2cnc3c(N)ncnc32)[C@H](O)[C@@H]1O]

In some embodiments, the A2 receptor agonist is binodenoson (WRC-0470) (described in Jacobson K A et al., 2006, Nature Reviews. Drug Discovery, 5 (3): 247-64), which has the following chemical structure:

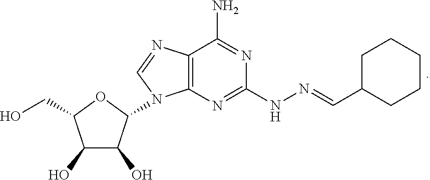 [SMILES: Nc1nc(N/N=C/C2CCCCC2)nc2c1ncn2[C@H]1O[C@@H](CO)[C@H](O)[C@@H]1O]

In some embodiments, the A2 receptor agonist is Sonedenoson (MRE-0094), which has the following chemical structure:

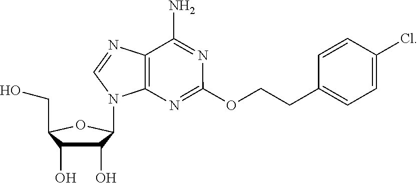

In some embodiments, the A2 receptor agonist is GW328267X, which has the following chemical structure:

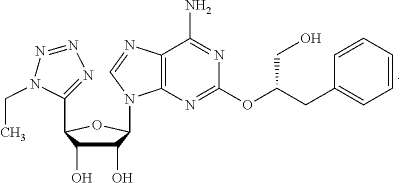

In some embodiments, the A2 receptor agonist is selected from the group consisting of adenosine, adenosine disodium triphosphate, Apadenoson, ATL1222, ATL-313, BVT.115959, Cardimax, Corvue (Binodenoson), GW328267X, Lexiscan, OPA-6566, PS246518, Selodenoson, and Sonedenoson.

Unless defined otherwise, all technical and scientific terms used herein have the same meaning as commonly understood by one skilled in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

The specific examples listed below are only illustrative and by no means limiting.

### EXAMPLES

### Example 1: Materials and Methods

Studies by the inventors of CD11c+ T-bet positive B cells in an experimental infection model system have identified a large number of genes that are differentially expressed by mouse CD11c⁺ T-bet⁺ B cells, relative to canonical naive B cells. Among these genes is Adora2A (adenosine A2A receptor), which encodes the adenosine 2A receptor (A2aR); Adora2A mRNA was found to be 10-fold higher in T-bet⁺ B cells, relative to canonical B cells. The inventors hypothesized, therefore, that inhibiting or stimulating the A2aR might modulate the activity of the T-bet⁺ B cells elicited in the infection model. Administration of an A2aR-stimulating antagonist, however, had no, or at most a modest, effect.

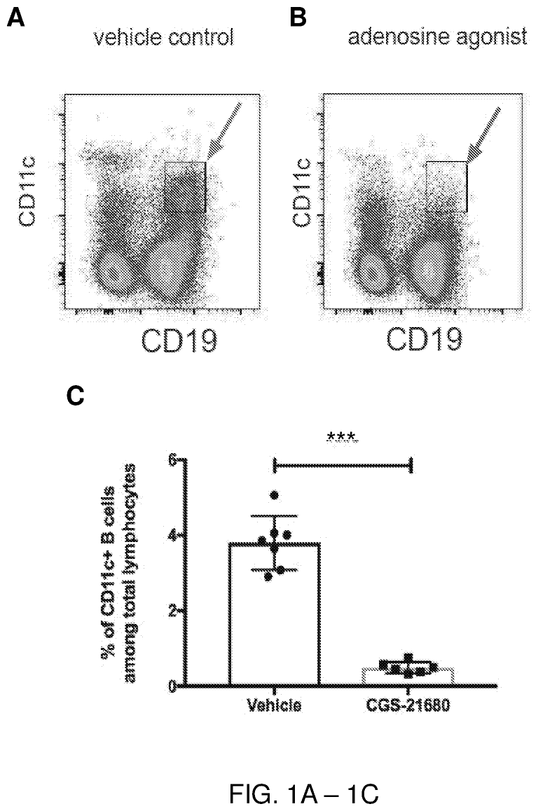

When antagonizing A2aR failed to provide the hypothesized effect, an A2aR agonist was tried. CGS21680, an A2aR agonist, was administered to mice 30 days following Ehrlichial infection, at a time T-bet⁺ CD11c⁺ B cells are permanently established (Ehrlichia is a genus of rickettsiales bacteria that is transmitted to vertebrates by ticks). Surprisingly, A2aR agonist administration caused a major reduction in the T-bet+ B cell population within 7 days of treatment (FIG. 1A-1C), suggesting that triggering of the A2aR has major effects on the targeted cells.

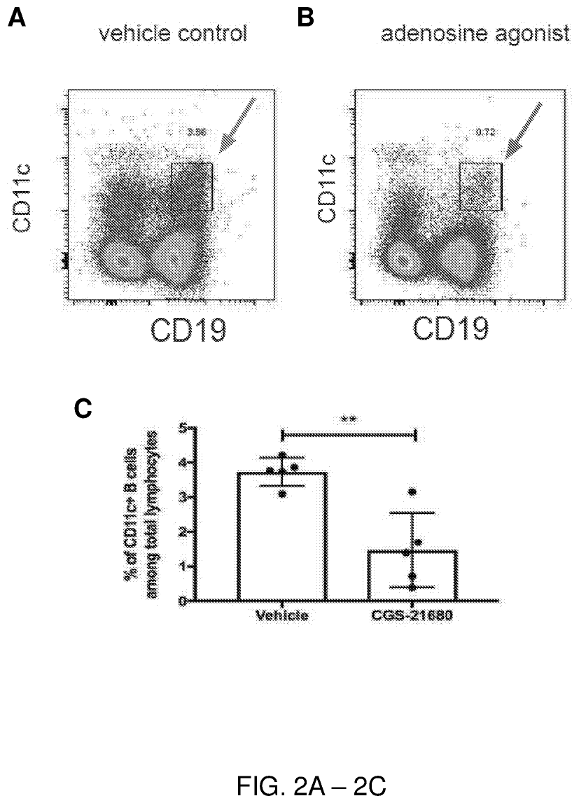

Further studies were performed to determine whether A2aR agonist treatment resulted in a prolonged depletion of the T-bet⁺ CD11c⁺ B cells. Mice were administered CGS21680, as in the studies shown in FIG. 1, but the cell analysis was delayed for 12 days. Significant cell depletion was observed after cessation of treatment (FIG. 2). These data indicate that even transient A2aR agonist treatment may lead to a permanent elimination or prolonged reduction of T-bet⁺ CD11c⁺ B cells in patients.

The data from these studies support the notion that this approach can be used to specifically target all T-bet⁺ B cells, including T-bet⁺ CD11c⁺ B cells, clinically, and suggest a novel and potentially important new therapy for a number of autoimmune diseases mediated in part by these pathogenic B cells.

An important aspect of this technology is that it targets a defined cell population that has been demonstrated to be essential for disease in animal models. Moreover, because the cell population is readily identifiable, using flow cytometric analysis of peripheral blood, the disclosed method can (1) assess whether a given patient would potentially benefit from the treatment, that is, they carry T-bet⁺ B cells, and (2) monitor the efficacy of the treatment on the targeted cell population, well in advance of clinical assessments of disease status. Thus, the present disclosure offers the possibility detecting and eliminating pathogenic B cells in autoimmune patients in real-time.

While there have been shown and described what are at present considered the preferred embodiments of the invention, those skilled in the art may make various changes and modifications which remain within the scope of the invention defined by the appended claims.

What is claimed is:

1. A method comprising:
(a) obtaining a biological sample from a subject, wherein the sample comprises B cells;
(b) detecting in the sample the presence of pathogenic B cells;
(c) administering to the subject a pharmaceutically effective dose of an Adora2A (adenosine A2A receptor) agonist (A2aR agonist) if pathogenic B cells are detected in step (b).

2. The method of claim 1, further comprising:
(d) obtaining, after the administering in step (c), an additional biological sample from a subject, wherein the additional biological sample comprises B cells;
(e) detecting in the additional biological sample the presence of pathogenic B cells; and
(f) administering to the subject a pharmaceutically effective dose of the A2aR agonist if pathogenic B cells are detected in step (e).

3. The method of claim 1, wherein the pathogenic B cells comprise T-bet⁺ B cells.

4. The method of claim 1, wherein the pathogenic B cells comprise CD11c⁺ cells

5. The method of claim 1, wherein the pathogenic B cells comprise T-bet⁺ and CD11c⁺ double-positive B cells or T-bet+ CD11c-negative single-positive cells.

6. The method of claim 1, wherein the detection of pathogenic B cells is achieved by flow cytometry.

7. The method of claim 1, wherein the biological sample is selected from peripheral blood, plasma, vitreous fluid, lymph fluid, synovial fluid, follicular fluid, seminal fluid, amniotic fluid, milk, whole blood, urine, cerebro-spinal fluid, saliva, sputum, tears, perspiration, and mucus.

8. The method of claim 1, wherein the A2aR agonist is selected from the group consisting of CGS21680, ATL-146e, YT-146, N6-(2-(3,5-dimethoxyphenyl)-2-(2-methylphenyl)ethyl)adenosine (DPMA), Regadenoson (CV-3146), UK-432,097, Limonene, Zeatin riboside, 5′-(N-Ethylcarboxamido)adenosine (NECA), binodenoson.

9. The method of claim 1, wherein the A2aR agonist has the following structure: 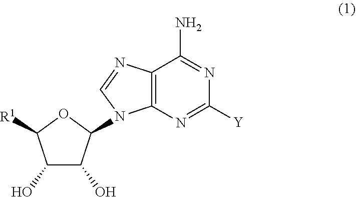
wherein:
R¹ is selected from the group consisting of —CH₂OH, —CO₂H, —CO₂R², —C(O)NR³R⁴, and —C(O)NHR⁴, wherein R², R³, and R⁴ are independently hydrocarbon groups containing 1-6 carbon atoms;
Y is selected from the group consisting of R⁵, —OR⁶, —NR⁷R⁸, —NHR⁸, —NH—N═CR⁹R¹⁰, and —NH—N═CHR¹⁰, wherein R⁵, R⁶, R⁷, R⁸, R⁹, and R¹⁰ are independently hydrocarbon groups containing 1-20 carbon atoms and optionally substituted with one or more heteroatoms selected from nitrogen and oxygen atoms.

10. The method of claim 9, wherein R¹ is —C(O)NHR⁴ or —C(O)NR³R⁴.

11. The method of claim 10, wherein R³ and R⁴ are independently hydrocarbon groups containing 1-4 carbon atoms.

12. The method of claim 10, wherein R³ and R⁴ are independently hydrocarbon groups containing 1-3 carbon atoms.

13. The method of claim 9, wherein Y is —NR⁷R⁸ or —NHR⁸.

14. The method of claim 13, wherein Y is —NHR⁸.

15. The method of claim 14, wherein R⁸ contains a five-membered or six-membered ring.

16. The method of claim 15, wherein R⁸ contains a benzene ring.

17. The method of claim 16, wherein R⁸ has the following structure: 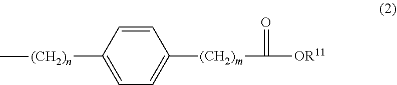
wherein:
n and m are independently integers of 1, 2, and 3; and
R¹¹ is a hydrogen atom or a hydrocarbon group containing 1-6 carbon atoms.

18. The method of claim 17, wherein the A2aR agonist has the following structure, corresponding to CGS21680: 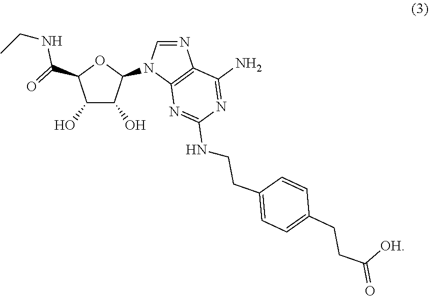 [SMILES: CCNC(=O)[C@H]1O[C@@H](n2cnc3c(N)nc(NCCc4ccc(CCC(=O)O)cc4)nc32)[C@H](O)[C@@H]1O]
